(19) United States
(12) Patent Application Publication
(10) Pub. No.: US 2017/0196583 A1
(43) Pub. Date: Jul. 13, 2017

(54) MEDICAL TREATMENT DEVICE

(71) Applicant: OLYMPUS CORPORATION, Tokyo (JP)

(72) Inventor: Yuta SUGIYAMA, Tokyo (JP)

(73) Assignee: OLYMPUS CORPORATION, Tokyo (JP)

(21) Appl. No.: 15/468,671

(22) Filed: Mar. 24, 2017

Related U.S. Application Data
(63) Continuation of application No. PCT/JP2014/079146, filed on Oct. 31, 2014.

(51) Int. Cl. A61B 17/32; A61B 18/04; A61B 18/12; A61B 18/08

(52) U.S. Cl. CPC A61B 17/320068; A61B 18/085; A61B 18/04; A61B 18/1206; A61B 2017/320076

(57) ABSTRACT

A medical treatment device includes: a probe where ultrasound vibration generated by each ultrasound transducer is transmitted from the one end to other end of the probe; a jaw portion configured to rotate around a central axis of the probe; and a controller configured to: calculate outputs, which respectively drive ultrasound transducers, based on a rotation angle of the jaw portion; and drive each of the ultrasound transducers by each of the calculated outputs. Each output is an output that sets a direction of vibration of the other end caused by ultrasound vibration generated by each of the ultrasound transducers to a direction from the central axis to the jaw portion with respect to a direction along the central axis.

### CROSS-REFERENCE TO RELATED APPLICATIONS

This application is a continuation of PCT international application Ser. No. PCT/JP2014/079146 filed on Oct. 31, 2014 which designates the United States, and the entire contents of the PCT international application is incorporated herein by reference.

### BACKGROUND

1. Technical Field

The disclosure relates to a medical treatment device.

2. Related Art

Conventionally, a medical treatment device which joins or anastomoses living tissues by using ultrasound vibration is known (for example, see JP 07-23972 A).

A medical treatment device described in JP 07-23972 A includes a pair of sandwiching parts that can be opened and closed, an ultrasound transducer that generates ultrasound vibration, and a vibration transmission member that transmits the ultrasound vibration generated by the ultrasound transducer to the pair of sandwiching parts. In the medical treatment device, living tissues are sandwiched by the pair of sandwiching parts and the living tissues are joined or anastomosed by transmitting an ultrasound vibration, which vibrates along a direction in which the pair of sandwiching parts faces each other, to the living tissues.

An extracellular matrix (collagen, elastin, or the like) of the living tissues is formed by a fibrous texture. Therefore, when the living tissues are joined together, the extracellular matrixes are extracted from the living tissues and the extracellular matrixes are closely entangled together, so that it is considered that the joining strength of the living tissues is improved. Further, when an ultrasound vibration is applied in a thickness direction of the living tissues, it is considered that the extracellular matrixes can be closely entangled together.

The medical treatment device described in JP 07-23972 A transmits the ultrasound vibration, which vibrates along a direction in which the pair of sandwiching parts that sandwiches the living tissues faces each other (a thickness direction of the living tissues), to the living tissues. Therefore, the extracellular matrixes extracted from the living tissues by the ultrasound vibration are closely entangled together by the ultrasound vibration. Thus, it is considered that the joining strength of the living tissues is improved.

### SUMMARY

In some embodiments, a medical treatment device includes: a vibration unit including a plurality of ultrasound transducers, each ultrasound transducer being configured to generate ultrasound vibration; a probe which extends linearly and where the vibration unit is attached to one end of the probe and the ultrasound vibration generated by each of the ultrasound transducers is transmitted from the one end to other end of the probe; a jaw portion configured to: sandwich living tissues between the jaw portion and the other end of the probe by moving relative to the probe; and rotate around a central axis of the probe; a rotation angle sensor configured to detect a rotation angle of the jaw portion around the central axis; and a controller configured to: calculate outputs, which respectively drive the ultrasound transducers, based on the rotation angle of the jaw portion; and drive each of the ultrasound transducers by each of the calculated outputs. Each output is an output that sets a direction of vibration of the other end caused by ultrasound vibration generated by each of the ultrasound transducers to a direction from the central axis to the jaw portion with respect to a direction along the central axis.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating a medical treatment device according to a first embodiment of the disclosure;

FIG. 2 is a cross-sectional view illustrating an internal structure of a treatment tool illustrated in FIG. 1;

FIG. 3 is a cross-sectional view illustrating an internal structure of the treatment tool illustrated in FIG. 1;

FIG. 4 is a cross-sectional view illustrating an internal structure of the treatment tool illustrated in FIG. 1;

FIG. 5A is a diagram illustrating an opening/closing action of a jaw portion illustrated in FIG. 1;

FIG. 5B is a diagram illustrating the opening/closing action of the jaw portion illustrated in FIG. 1;

FIG. 6A is a diagram illustrating a rotating action of the jaw portion illustrated in FIG. 1;

FIG. 6B is a diagram illustrating the rotating action of the jaw portion illustrated in FIG. 1;

FIG. 7A is a diagram illustrating a reference position of the jaw portion when a rotation angle is detected by a rotation angle sensor illustrated in FIG. 2;

FIG. 7B is a diagram illustrating the reference position of the jaw portion when the rotation angle is detected by the rotation angle sensor illustrated in FIG. 2;

FIG. 8 is a block diagram illustrating a configuration of a controller and a foot switch illustrated in FIG. 1;

FIG. 9 is a flowchart illustrating a joining control performed by the controller illustrated in FIG. 8;

FIG. 10A is a diagram schematically illustrating horizontal vibration generated in a probe by step S4 illustrated in FIG. 9;

FIG. 10B is a diagram schematically illustrating horizontal vibration generated in the probe by step S4 illustrated in FIG. 9;

FIG. 11 is a diagram illustrating a modified example 1-1 of the first embodiment of the disclosure;

FIG. 12 is a diagram illustrating a modified example 1-2 of the first embodiment of the disclosure;

FIG. 13 is a diagram illustrating a modified example 1-3 of the first embodiment of the disclosure;

FIG. 14 is a flowchart illustrating a joining control according to a second embodiment of the disclosure;

FIG. 15 is a diagram schematically illustrating horizontal vibration generated in the probe by steps S8 and S12 illustrated in FIG. 14;

FIG. 16 is a diagram schematically illustrating a treatment tool according to a third embodiment of the disclosure;

FIG. 17 is a diagram schematically illustrating the treatment tool according to the third embodiment of the disclosure;

FIG. 18 is a block diagram illustrating a configuration of a controller in a medical treatment device according to a fourth embodiment of the disclosure;

FIG. 19 is a block diagram illustrating a configuration of a controller in a medical treatment device according to a fifth embodiment of the disclosure;

FIG. 20 is a diagram illustrating a modified example of the first to the fifth embodiments of the disclosure; and

FIG. 21 is a diagram illustrating the modified example of the first to the fifth embodiments of the disclosure.

### DETAILED DESCRIPTION

Hereinafter, embodiments for implementing the disclosure (hereinafter referred to as embodiments) will be described with reference to the drawings. The disclosure is not limited by the embodiments described below. Further, in the description of the drawings, the same components are denoted by the same reference numerals.

### First Embodiment

Schematic Configuration of Medical Treatment Device

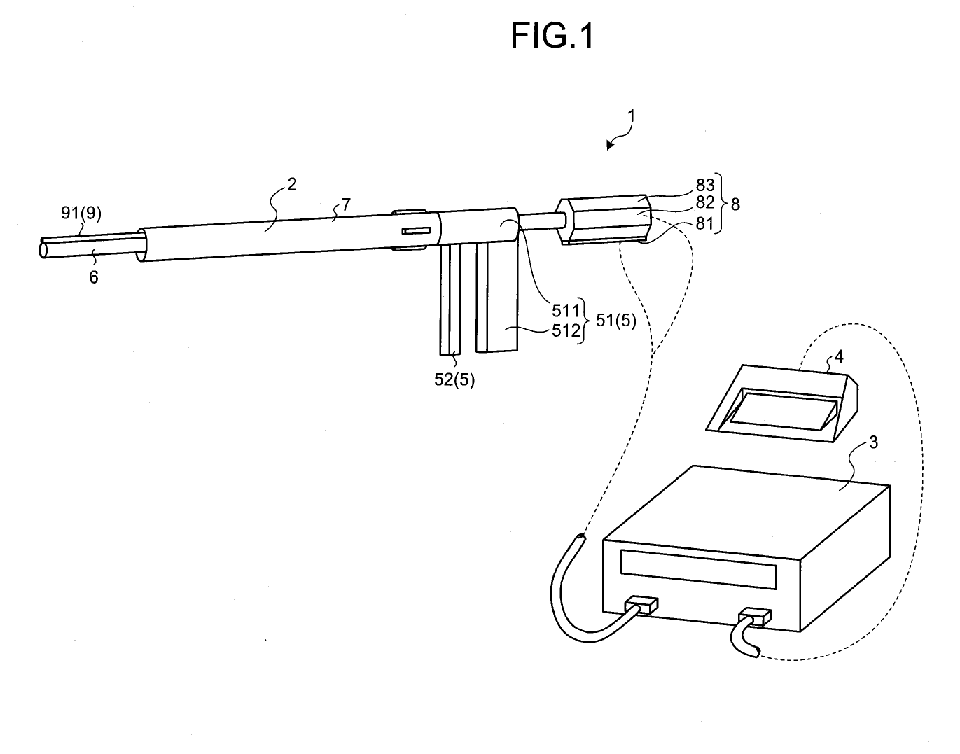

FIG. 1 is a diagram schematically illustrating a medical treatment device 1 according to a first embodiment of the disclosure.

The medical treatment device 1 treats (joins or anastomoses) living tissues to be treated by using ultrasound vibration. As illustrated in FIG. 1, the medical treatment device 1 includes a treatment tool 2, a controller 3, and a foot switch 4.

Configuration of Treatment Tool

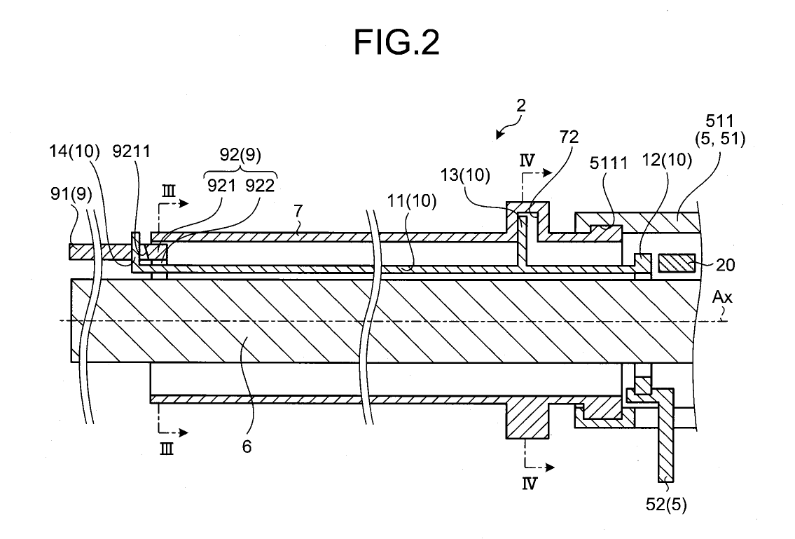

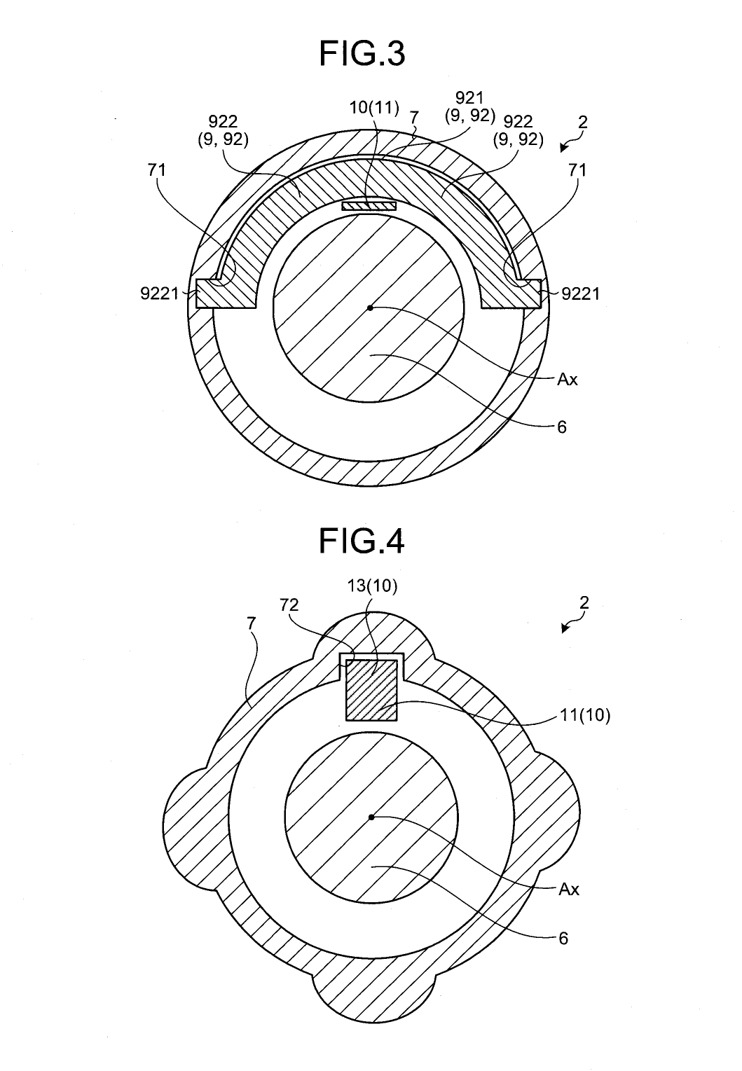

FIGS. 2 to 4 are cross-sectional views illustrating an internal structure of the treatment tool 2. Specifically, FIG. 2 is a vertical cross-sectional view taken along a plane including a central axis Ax of a probe 6. FIG. 3 is a horizontal cross-sectional view of the treatment tool 2 taken along line illustrated in FIG. 2. FIG. 4 is a horizontal cross-sectional view of the treatment tool 2 taken along line IV-IV illustrated in FIG. 2. FIGS. 2 to 4 illustrate a portion more forward than an operation lever 52 (a portion including the left end portion in FIG. 1) and omit illustration of a part of a handle 5 and a vibration unit 8.

The treatment tool 2 is, for example, a linear-type surgical medical treatment tool for performing treatment on living tissues through an abdomen wall. As illustrated in FIGS. 1 to 4, the treatment tool 2 includes the handle 5 (FIGS. 1 and 2), the probe 6, an outer cylinder 7, the vibration unit 8 (FIG. 1), a jaw portion 9 (FIGS. 1 to 3), an open and close transmission member 10 (FIGS. 2 to 4), and a rotation angle sensor 20 (FIG. 2). The central axis Ax of the probe 6 is a central axis in the longitudinal direction of the probe 6.

The handle 5 is a portion which the operator holds. As illustrated in FIG. 1 or FIG. 2, the handle 5 includes an outer frame 51 and an operation lever 52.

The outer frame 51 includes a cylindrical portion 511 that has a cylindrical shape and a held portion 512 (FIG. 1) that is formed integrally with the cylindrical portion 511 and is held by the operator.

As illustrated in FIG. 2, a ring-shaped support recess portion 5111 extending along a circumferential direction around an axis of the cylindrical portion 511 is formed on an inner circumferential surface of the cylindrical portion 511.

The operation lever 52 is a portion operated by the operator and is supported by the cylindrical portion 511 movably along the central axis Ax.

As illustrated in FIGS. 1 to 4, the probe 6 has a linearly extending columnar shape, is inserted into the cylindrical portion 511, and is supported by the cylindrical portion 511 (the handle 5) in a state in which both ends are exposed to the outside. The vibration unit 8 is attached to one end (the right end portion in FIG. 1) of the probe 6, and the probe 6 transmits ultrasound vibration generated by the vibration unit 8 from the one end to the other end (the left end portion in FIG. 1).

The outer cylinder 7 is a portion that is operated by the operator and, as illustrated in FIGS. 1 to 4, has a substantially cylindrical shape into which the probe 6 can be inserted. As illustrated in FIG. 2, the outer cylinder 7 is formed so that the outer diameter size of one end (the right end portion in FIG. 2) is greater than the outer diameter size of the other portion. As illustrated in FIG. 2, the one end of the outer cylinder 7 is engaged with the support recess portion 5111 and can be rotated around the central axis Ax according to an operation by the operator.

As illustrated in FIG. 3, on an inner circumferential surface on the other end side of the outer cylinder 7, a pair of bearing recess portions 71 is formed, which is located on a plane including the central axis Ax and which has a circular shape in cross-section and faces each other with the central axis Ax therebetween.

Further, as illustrated in FIG. 2, on an inner circumferential surface on the one end side of the outer cylinder 7, an engaging recess portion 72 is formed, which engages with the open and close transmission member 10.

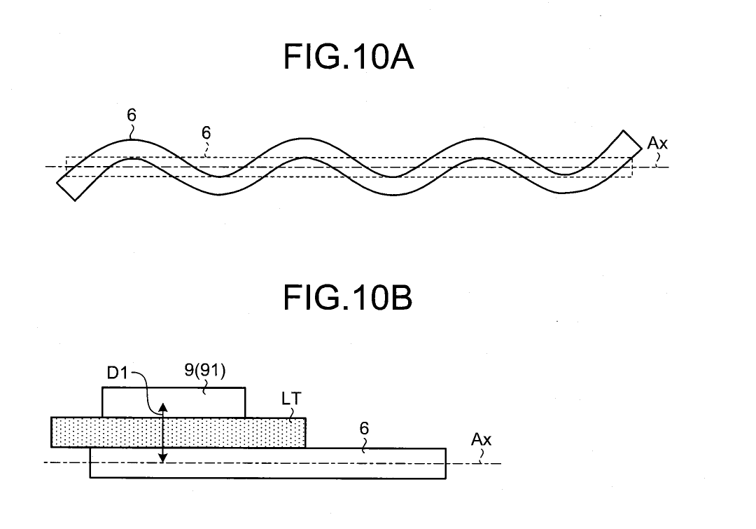

The vibration unit 8 generates ultrasound vibration and causes the probe 6 to generate horizontal vibration (see FIG. 10A). As illustrated in FIG. 1, the vibration unit 8 includes a first and a second ultrasound transducers 81 and 82 and a horizontal vibration enlargement unit 83.

The first and the second ultrasound transducers 81 and 82 have the same configuration. In the first embodiment, each of the first and the second ultrasound transducers 81 and 82 is formed by a piezoelectric transducer using a piezoelectric element that expands and contracts when an AC voltage is applied.

The horizontal vibration enlargement unit 83 is a member that enlarges the ultrasound vibration (amplitude) generated by the first and the second ultrasound transducers 81 and 82. As illustrated in FIG. 1, the horizontal vibration enlargement unit 83 is formed by a regular octagonal column whose outer diameter size is greater than that of the probe 6. The horizontal vibration enlargement unit 83 is attached to one end of the probe 6 so that a column axis corresponds to the central axis Ax and a pair of side surfaces facing each other is perpendicular to a vertical direction (vertical axis) in FIG. 2.

The resonance frequency of the horizontal vibration enlargement unit 83 is substantially the same as the resonance frequency of the horizontal vibration of the probe 6 and is, for example, 40 kHz.

Here, the first and the second ultrasound transducers 81 and 82 are attached to two side surfaces, which are 90° shifted from each other around the central axis Ax when observed from a direction along the central axis Ax, among eight side surfaces of the horizontal vibration enlargement unit 83. More specifically, the first ultrasound transducer 81 is attached to a side surface located below in FIG. 1. The second ultrasound transducer 82 is attached to a side surface located right in FIG. 1 when observed from a distal end side of the treatment tool 2.

The first and the second ultrasound transducers 81 and 82 are electrically attached to the controller 3 through an electrical cable C, and an AC voltage (whose frequency is the same as the resonance frequency of the horizontal vibration of the probe 6) is applied to the first and the second ultrasound transducers 81 and 82 under control of the controller 3, so that the first and the second ultrasound transducers 81 and 82 expand and contract in a direction along the central axis Ax. In other words, in the first embodiment, the first and the second ultrasound transducers 81 and 82 are configured to generate the horizontal vibration (the ultrasound vibration). The horizontal vibration generated by the first and the second ultrasound transducers 81 and 82 is enlarged by the horizontal vibration enlargement unit 83 and causes the probe 6 to generate horizontal vibration through the horizontal vibration enlargement unit 83.

The jaw portion 9 performs an opening/closing action on the other end of the probe 6 according to an operation (hereinafter referred to as an opening/closing operation) on the operation lever 52 performed by the operator. Further, the jaw portion 9 performs a rotating action around the central axis Ax according to an operation (hereinafter referred to as a rotating operation) on the outer cylinder 7 performed by the operator. As illustrated in FIGS. 1 to 3, the jaw portion 9 includes a jaw portion main body 91 (FIGS. 1 and 2) and a jaw portion side engaging portion 92 (FIGS. 2 and 3).

The jaw portion main body 91 has an arc shape in cross-section following the outer circumferential surface of the probe 6 and is formed by a plate-like member extending along the central axis Ax. The jaw portion main body 91 and the probe 6 sandwich living tissues by an opening/closing action of the jaw portion 9.

The jaw portion side engaging portion 92 is formed integrally with the jaw portion main body 91 and is a portion that engages with each of the open and close transmission member 10 and the outer cylinder 7. As illustrated in FIGS. 2 and 3, the jaw portion side engaging portion 92 includes a jaw portion side first engaging portion 921 and a pair of jaw portion side second engaging portions 922.

The jaw portion side first engaging portion 921 is formed integrally with the jaw portion main body 91 and has the same shape (a plate-like member having an arc shape in cross-sectional view) as that of the jaw portion main body 91.

As illustrated in FIG. 2, an engaging hole 9211 which penetrates the jaw portion side first engaging portion 921 and engages with the open and close transmission member 10 is formed in the jaw portion side first engaging portion 921.

Each of the pair of jaw portion side second engaging portions 922 is integrally formed at one end of the jaw portion side first engaging portion 921 (the right end portion in FIG. 2). As illustrated in FIG. 3, the pair of jaw portion side second engaging portions 922 extends along a rotation direction around the central axis Ax from the one end in directions in which the pair of jaw portion side second engaging portions 922 becomes away from each other, and each of the pair of jaw portion side second engaging portions 922 has an arc shape whose central angle is substantially 90°.

As illustrated in FIG. 3, a pair of engaging pins 9221 protruding to the outside (side being away from the central axis Ax) is respectively formed at the distal end portions (portions separate from the first engaging portion 921) of the pair of jaw portion side second engaging portions 922. The pair of engaging pins 9221 respectively engages with the pair of bearing recess portions 71, so that the jaw portion 9 can rotate around the pair of engaging pins 9221 and the pair of bearing recess portions 71. In other words, the jaw portion 9 enables an opening/closing action on the other end of the probe 6 by the engagement.

The open and close transmission member 10 is arranged inside the outer cylinder 7 and causes the jaw portion 9 to perform an opening/closing action according to an opening/closing operation. As illustrated in FIGS. 2 to 4, the open and close transmission member 10 includes a long portion 11, an annular portion 12 (FIG. 2), a transmission side first engaging portion 13 (FIGS. 2 and 4), and a transmission side second engaging portion 14 (FIG. 2).

The long portion 11 is formed by a long flat plate extending along the central axis Ax.

The annular portion 12 is integrally formed with one end (the right end portion in FIG. 2) of the long portion 11 and has a circular ring shape through which the probe 6 can be inserted. As illustrated in FIG. 2, the annular portion 12 is connected to the operation lever 52 in a state in which the annular portion 12 can be rotated around the central axis Ax.

The transmission side first engaging portion 13 is formed by a flat plate having a rectangular shape in plan view and is integrally formed with the long portion 11 on the upper surface of the long portion 11 in FIG. 2 or FIG. 4 in a posture where a surface of the flat plate is perpendicular to a line in parallel with the central axis Ax. As illustrated in FIG. 2 or FIG. 4, the transmission side first engaging portion 13 is inserted into the engaging recess portion 72.

Here, the length dimension in the horizontal direction of the transmission side first engaging portion 13 in FIG. 4 is formed a little smaller than the dimension in the horizontal direction of the engaging recess portion 72. Further, as illustrated in FIG. 2, the thickness dimension (the length dimension in the horizontal direction (the direction along the central axis Ax) in FIG. 2) of the transmission side first engaging portion 13 is formed a little smaller than the dimension in the horizontal direction of the engaging recess portion 72. More specifically, the dimension of a gap between the transmission side first engaging portion 13 and the engaging recess portion 72 (a gap in a direction along the central axis Ax) is set to be substantially the same as a movable range of reciprocating movement of the operation lever 52 along the central axis Ax.

The transmission side second engaging portion 14 is formed by a flat plate having a rectangular shape in plan view and is integrally formed with the other end (the left end portion in FIG. 2) of the long portion 11 so as to be protruded from the upper surface of the long portion 11 in FIG. 2 in a posture where a surface of the flat plate is perpendicular to a line in parallel with the central axis Ax. As illustrated in FIG. 2, the transmission side second engaging portion 14 is inserted into the engaging hole 9211.

Opening/Closing Action of Jaw Portion

Next, the opening/closing action of the jaw portion 9 described above will be de described.

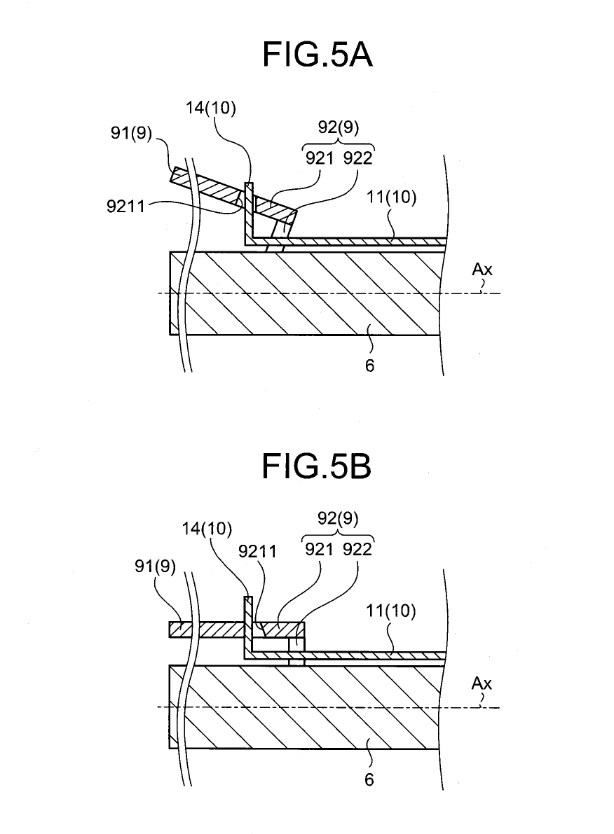

FIGS. 5A and 5B are diagrams illustrating an opening/closing action of the jaw portion 9. Specifically, FIGS. 5A and 5B are cross-sectional views corresponding to FIG. 2.

When the operation lever 52 moves to the right in FIG. (to the right in FIG. 5A), the open and close transmission member 10 moves to the right in FIG. 5A along with the operation lever 52 along the central axis Ax due to a connection structure between the annular portion 12 and the operation lever 52 described above and an engaging structure between the transmission side first engaging portion 13 and the engaging recess portion 72 described above. At this time, the transmission side second engaging portion 14 presses an edge portion of the engaging hole 9211 to the right in FIG. 5A. By this pressure, as illustrated in FIG. 5A, the jaw portion 9 rotates around the pair of engaging pins 9221 and the pair of bearing recess portions 71 (FIG. 3) in a direction separating away from the other end of the probe 6.

On the other hand, when the operation lever 52 moves to the left in FIG. 2 (to the left in FIG. 5B), the open and close transmission member 10 moves to the left in FIG. 5B along with the operation lever 52 along the central axis Ax due to the connection structure between the annular portion 12 and the operation lever 52 described above and the engaging structure between the transmission side first engaging portion 13 and the engaging recess portion 72 described above. At this time, the transmission side second engaging portion 14 presses an edge portion of the engaging hole 9211 to the left in FIG. 5B. By this pressure, as illustrated in FIG. 5B, the jaw portion 9 rotates around the pair of engaging pins 9221 and the pair of bearing recess portions 71 (FIG. 3) in a direction approaching the other end of the probe 6. As a result, the treatment tool 2 can sandwich living tissues between the jaw portion 9 and the other end of the probe 6.

Rotating Action of Jaw Portion

Next, the rotating action of the jaw portion 9 described above will be described.

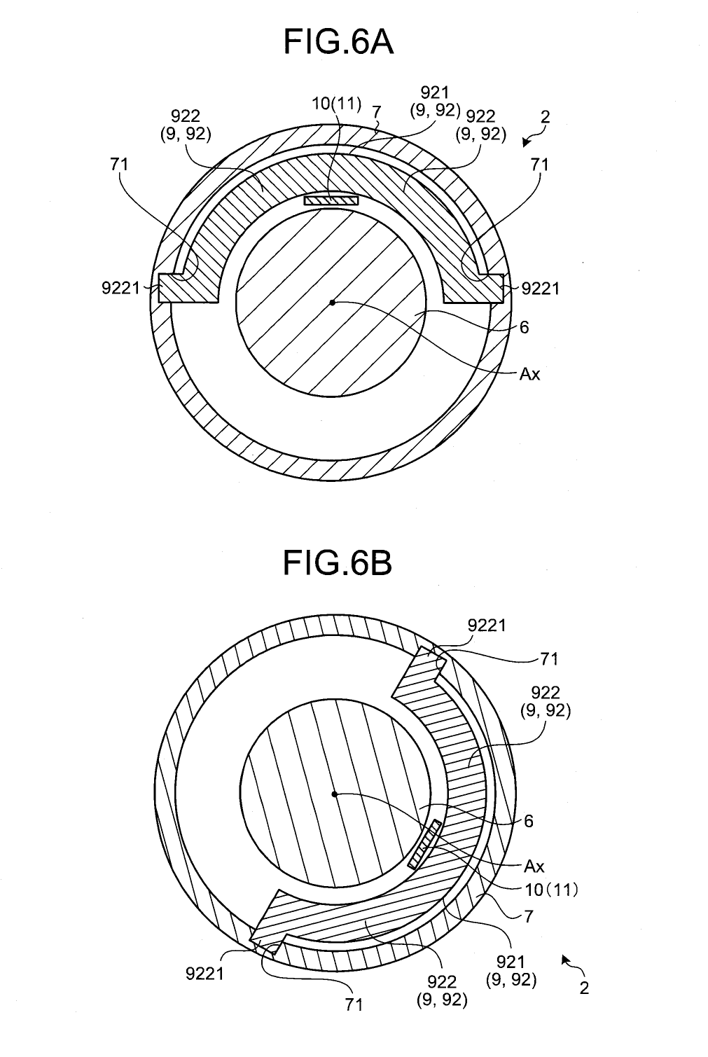

FIGS. 6A and 6B are diagrams illustrating the rotating action of the jaw portion 9. Specifically, FIGS. 6A and 6B are cross-sectional views corresponding to FIG. 3.

When the outer cylinder 7 is rotated around the central axis Ax by a rotating operation from a state illustrated in FIG. 6A, the jaw portion 9 rotates around the central axis Ax along with the outer cylinder 7 as illustrated in FIG. 6B because the pair of engaging pins 9221 respectively engages with the pair of bearing recess portions 71. At this time, in the same manner, as illustrated in FIG. 6B, the open and close transmission member 10 also rotates around the central axis Ax along with the outer cylinder 7 and the jaw portion 9 due to the connection structure between the annular portion 12 and the operation lever 52 described above and the engaging structure between the transmission side first engaging portion 13 and the engaging recess portion 72 described above.

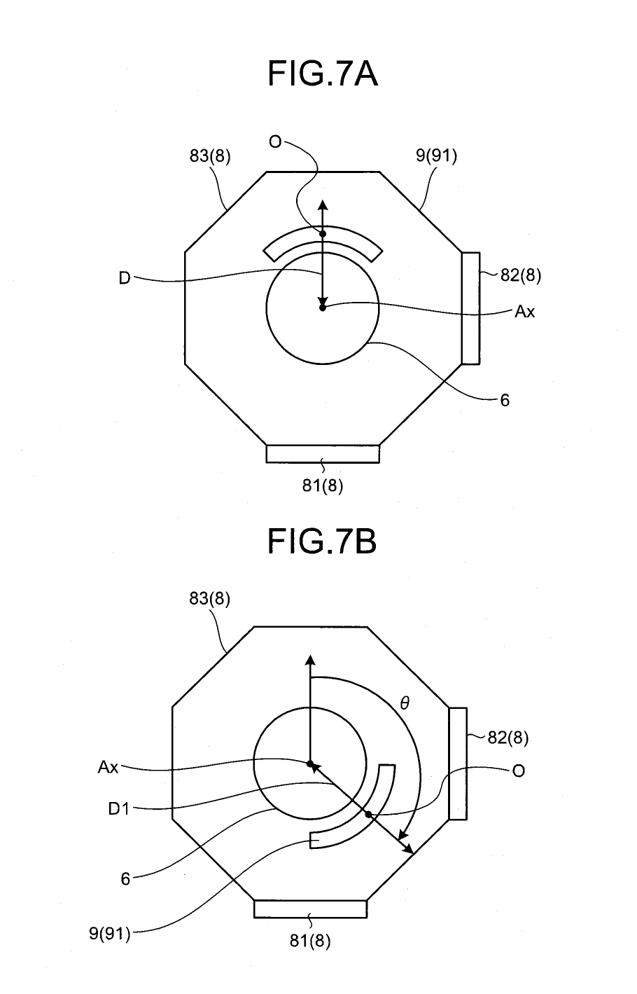

FIGS. 7A and 7B are diagrams illustrating a reference position of the jaw portion 9 when the rotation angle sensor 20 detects a rotation angle θ. Specifically, FIGS. 7A and 7B are schematic diagrams of the probe 6, the vibration unit 8, and the jaw portion 9 (the jaw portion main body 91) when observed from the distal end side of the treatment tool 2 along the central axis Ax.

Here, the rotation angle sensor 20 is formed from a rotary encoder or the like and detects the rotation angle θ (FIG. 7B) around the central axis Ax in the open and close transmission member 10 (the jaw portion 9). The rotation angle sensor 20 outputs a signal according to the detected rotation angle θ to the controller 3.

The reference position of the jaw portion 9 when the rotation angle sensor 20 detects the rotation angle θ faces the first ultrasound transducer 81 as illustrated in FIG. 7A and is a position of the jaw portion 9 in a case in which when horizontal vibration is generated in the probe 6 by ultrasound vibration generated by the first ultrasound transducer 81, a vibration direction D of the horizontal vibration corresponds to a direction from the central axis Ax to a center position O of the jaw portion 9 (the jaw portion main body 91) (a center position in the width direction (the horizontal direction in FIG. 7A) of the jaw portion main body 91).

Configuration of Controller and Foot Switch

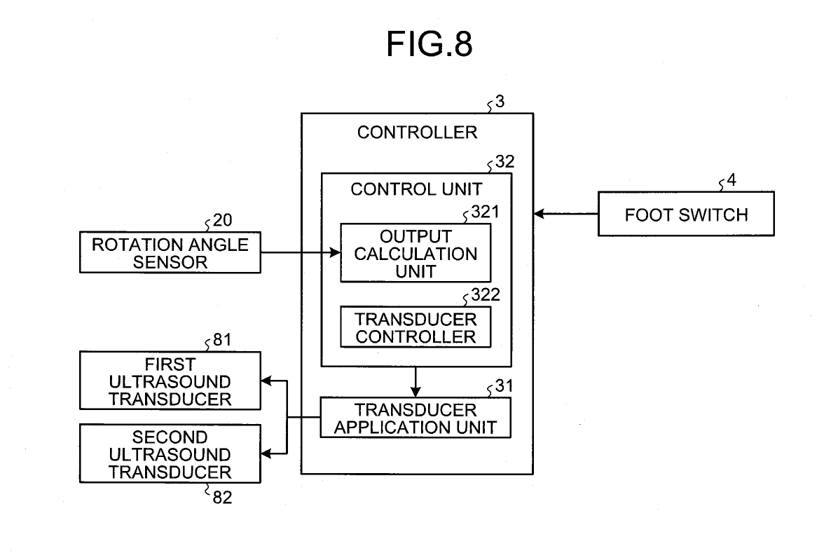

FIG. 8 is a block diagram illustrating a configuration of the controller 3 and the foot switch 4.

As a configuration of the controller 3, FIG. 8 mainly illustrates an essential part of the disclosure.

The foot switch 4 is a portion operated by a foot of the operator. The controller 3 starts joining control described later according to the operation (ON) to the foot switch 4.

A means to start the joining control is not limited to the foot switch 4, and a switch operated by a hand or the like may be employed.

The controller 3 integrally controls action of the treatment tool 2. As illustrated in FIG. 8, the controller 3 includes a transducer application unit 31 and control unit 32.

The transducer application unit 31 applies an AC voltage (whose frequency is the same as the resonance frequency of the horizontal vibration of the probe 6) to the first and the second ultrasound transducers 81 and 82 through the electrical cable C by each first output calculated by the control unit 32 under control of the control unit 32. That is to say, the transducer application unit 31 has a function as a vibration drive unit according to the disclosure.

The control unit 32 includes a CPU (Central Processing Unit) and the like, and when the foot switch 4 turns ON, the control unit 32 performs the joining control according to a predetermined control program. As illustrated in FIG. 8, the control unit 32 includes an output calculation unit 321 and a transducer controller 322.

The output calculation unit 321 calculates each first output that drives each of the first and the second ultrasound transducers 81 and 82 based on the rotation angle θ detected by the rotation angle sensor 20.

The transducer controller 322 drives the transducer application unit 31 and applies an AC voltage to the first and the second ultrasound transducers 81 and 82 from the transducer application unit 31 through the electrical cable C by each first output calculated by the output calculation unit 321.

Action of Medical Treatment Device

Next, an action of the medical treatment device 1 described above will be described.

In the description below, the joining control performed by the control unit 32 will be mainly described as the action of the medical treatment device 1.

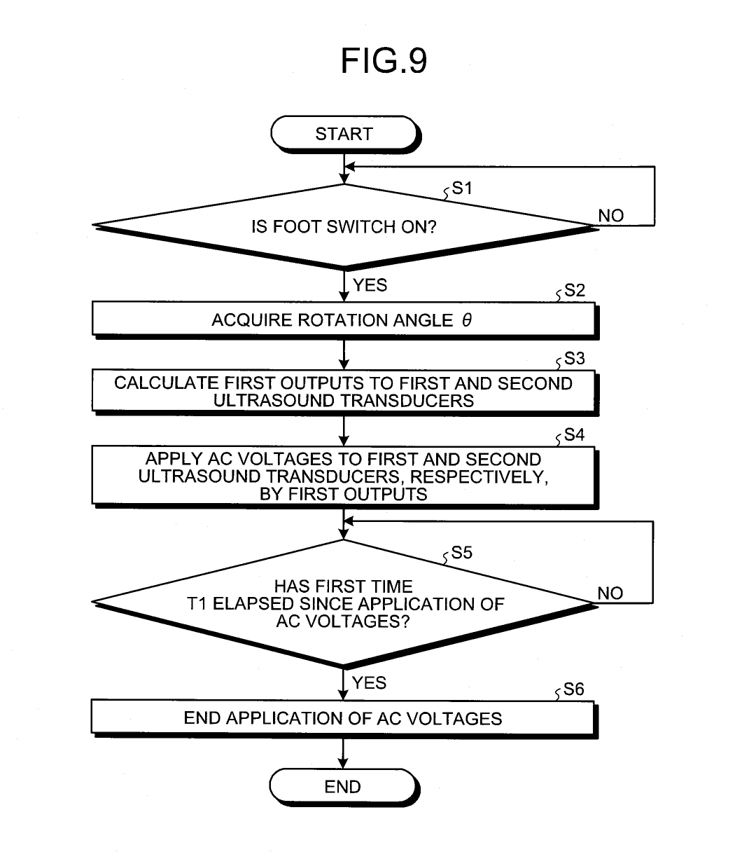

FIG. 9 is a flowchart illustrating the joining control performed by the control unit 32.

The operator holds the treatment tool 2 and inserts the distal end portion of the treatment tool 2 into, for example, an abdominal cavity through an abdominal wall. Then, the operator operates the operation lever 52, opens and closes a gap between the other end of the probe 6 and the jaw portion 9 (the jaw portion main body 91), and sandwiches living tissues LT to be treated with the other end of the probe 6 and the jaw portion 9 (the jaw portion main body 91) (see FIG. 10B).

Thereafter, the operator operates (turns ON) the foot switch 4 and causes the controller 3 to start the joining control.

When the foot switch 4 turns ON (step S1: Yes), the output calculation unit 321 acquires the rotation angle θ detected by the rotation angle sensor 20 (step S2).

After step S2, the output calculation unit 321 calculates a first output Va1 to the first ultrasound transducer 81 and a first output Vb1 to the second ultrasound transducer 82 from the following formulas (1) and (2) by using the rotation angle θ (step S3).

Va1=Vo×cos θ (1)

Vb1=Vo×sin θ (2)

Here, in the above formulas (1) and (2), Vo is an output voltage required by one ultrasound transducer to realize an arbitrary vibration amplitude S at the other end of the probe 6.

After step S3, the transducer controller 322 drives the transducer application unit 31 and causes the transducer application unit 31 to apply AC voltages to the first and the second ultrasound transducers 81 and 82, respectively, by the first outputs Va1 and Vb1 (step S4).

FIGS. 10A and 10B are diagrams schematically illustrating horizontal vibration generated in the probe 6 by step S4. Specifically, FIG. 10A illustrates with a solid line the probe 6 where the horizontal vibration is generated and illustrates with a dashed line the probe 6 where the horizontal vibration is not generated. FIG. 10B illustrates a relationship between a vibration direction D1 of the other end of the probe 6 and the living tissues LT.

When the AC voltages are applied to the first and the second ultrasound transducers 81 and 82, respectively, by the first outputs Va1 and Vb1, the respective first and the second ultrasound transducers 81 and 82 generates ultrasound vibration. Then, as illustrated in FIG. 10A, horizontal vibration is generated in the probe 6 by the ultrasound vibration generated by the respective first and the second ultrasound transducers 81 and 82. At this time, the vibration direction D1 of the horizontal vibration (the vibration direction D1 of the other end of the probe 6) is set to a direction from the central axis Ax to the jaw portion 9 as illustrated in FIG. 10B regardless of the rotation angle θ of the jaw portion 9. More specifically, the vibration direction D1 is set to a direction from the central axis Ax to the center position O of the jaw portion 9 (a first direction) with respect to a direction along the central axis Ax regardless of the rotation angle θ of the jaw portion 9 (FIG. 7B).

That is to say, each of the first outputs Va1 and Vb1 is an output that sets the vibration direction D1 of the other end of the probe 6 to the first direction with respect to a direction along the central axis Ax.

Subsequently, the transducer controller 322 monitors at all times whether or not a first time T1 has elapsed since the application of AC voltages in step S4 (step S5).

When determining that the first time T1 has elapsed (step S5: Yes), the transducer controller 322 stops the drive of the transducer application unit 31 (ends the application of AC voltages to the first and the second ultrasound transducers 81 and 82) (step S6).

The living tissues LT are joined by the processing described above.

In the medical treatment device 1 according to the first embodiment described above, the jaw portion 9 performs the opening/closing action according to the opening/closing operation and performs the rotating action according to the rotating operation. Therefore, the operator can sandwich the living tissues LT with the jaw portion 9 and the probe 6 from various directions by only performing the rotating operation without changing the posture of the medical treatment device 1 itself.

The medical treatment device 1 calculates the first outputs Va1 and Vb1 that set the vibration direction D1 of the other end of the probe 6 to the first direction (the direction from the central axis Ax to the center position O of the jaw portion 9) with respect to a direction along the central axis Ax, based on the rotation angle θ of the jaw portion 9. Then, the medical treatment device 1 generates horizontal vibration in the probe 6 by applying an AC voltage to the first and the second ultrasound transducers 81 and 82 by the first outputs Va1 and Vb1. Therefore, it is possible to set the vibration direction D1 to the first direction regardless of the rotation angle θ of the jaw portion 9. In other words, regardless of the rotation angle θ of the jaw portion 9, it is possible to closely entangle the extracellular matrixes extracted from the living tissues LT by the horizontal vibration of the probe 6, so that it is possible to improve the joining strength of the living tissues LT.

As described above, according to the medical treatment device 1 of the first embodiment, an effect is obtained that it is possible to improve the operability and also improve the joining strength of the living tissues LT.

### Modified Example 1-1 of First Embodiment

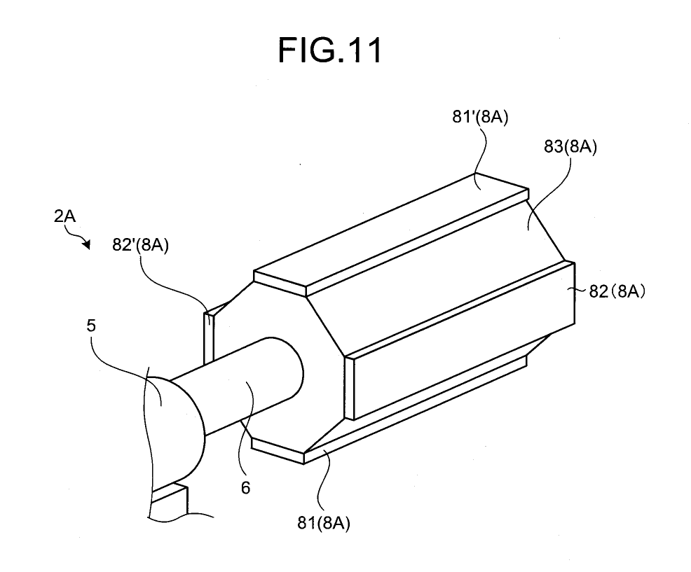

FIG. 11 is a diagram illustrating a modified example 1-1 of the first embodiment of the disclosure. Specifically, FIG. 11 is an enlarged schematic diagram of a part (one end side of the probe 6) of a treatment tool 2A according to the modified example 1-1.

In the first embodiment described above, in the vibration unit 8, only the first and the second ultrasound transducers 81 and 82 are attached to the horizontal vibration enlargement unit 83. However, it is not limited to this.

For example, as in a vibration unit 8A (FIG. 11) according to the modified example 1-1, it is possible to employ a configuration in which two first ultrasound transducers 81 and 81′ and two second ultrasound transducers 82 and 82′ are attached to the horizontal vibration enlargement unit 83.

Here, the first ultrasound transducer 81′ has the same configuration as that of the first ultrasound transducer 81 and is attached to a side surface facing a side surface where the first ultrasound transducer 81 is attached (a lower side surface in FIGS. 1 and 11) among the eight side surfaces of the horizontal vibration enlargement unit 83.

The first ultrasound transducer 81′ is applied with an AC voltage whose phase is opposite to that of an AC voltage applied to the first ultrasound transducer 81 by the first output Va1 under control of the controller 3.

Further, the second ultrasound transducer 82′ has the same configuration as that of the second ultrasound transducer 82 and is attached to a side surface facing a side surface where the second ultrasound transducer 82 is attached (a right side surface in FIGS. 1 and 11 with respect to a direction along the central axis Ax (when observed from the distal end side of the treatment tool 2A)) among eight side surfaces of the horizontal vibration enlargement unit 83.

The second ultrasound transducer 82′ is applied with an AC voltage whose phase is opposite to that of an AC voltage applied to the second ultrasound transducer 82 by the first output Vb1 under control of the controller 3.

Therefore, even when the vibration unit 8A as described in the modified example 1-1 is employed, it is possible to perform the same joining control as the joining control (FIG. 9) explained in the first embodiment described above except that the AC voltages applied to the first ultrasound transducers 81 and 81′ (the second ultrasound transducers 82 and 82′) have phases opposite to each other.

As described above, by increasing the number of ultrasound transducers, it is possible to increase power of the horizontal vibration in the probe 6.

### Modified Example 1-2 of First Embodiment

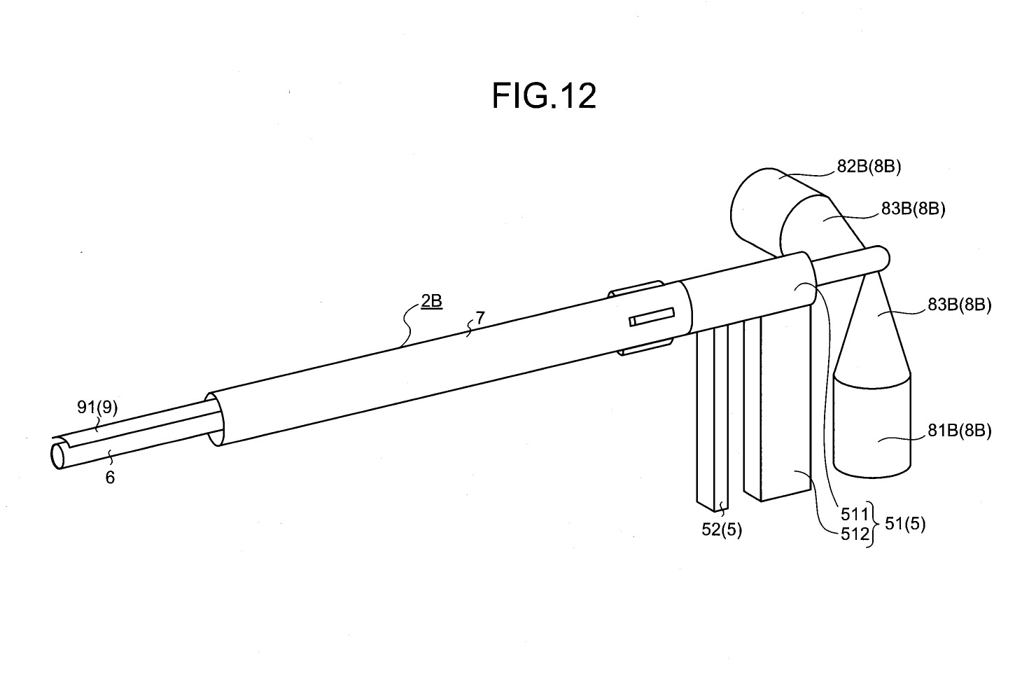

FIG. 12 is a diagram illustrating a modified example 1-2 of the first embodiment of the disclosure. Specifically, FIG. 12 is a diagram schematically illustrating a treatment tool 2B according to the modified example 1-2.

In the first embodiment described above, when AC voltages are respectively applied to the first and the second ultrasound transducers 81 and 82, the first and the second ultrasound transducers 81 and 82 generate horizontal vibration (ultrasound vibration). However, it is not limited to this.

For example, like the treatment tool 2B in the modified example 1-2 (FIG. 12), it is possible to employ a configuration in which a vibration unit 8B is employed instead of the vibration unit 8.

Specifically, as illustrated in FIG. 12, the vibration unit 8B includes a first and a second ultrasound transducers 81B and 82B and two vertical vibration enlargement units 83B.

The two vertical vibration enlargement units 83B are members that enlarge the ultrasound vibration (amplitude) generated by the first and the second ultrasound transducers 81B and 82B. The two vertical vibration enlargement units 83B have the same truncated cone shape and the smaller diameter side (the upper base) of each truncated cone shape is attached to one end of the probe 6 in a posture in which the central axis of the truncated cone is perpendicular to the central axis Ax. More specifically, one vertical vibration enlargement unit 83B is attached under the probe 6 in FIG. 12. In other words, the one vertical vibration enlargement unit 83B is attached to the one end of the probe 6 in a posture in which the central axis of the truncated cone is along the vertical direction in FIG. 12. On the other hand, the other vertical vibration enlargement unit 83B is attached to the one end of the probe 6 at a position 90° shifted from the one vertical vibration enlargement unit 83B around the central axis Ax (at a position in the left in FIG. 12 when observed from the distal end side of the treatment tool 2B).

Here, the resonance frequency of the two vertical vibration enlargement units 83B is substantially the same as the resonance frequency of the horizontal vibration of the probe 6 and is, for example, 40 kHz.

The first and the second ultrasound transducers 81B and 82B have the same configuration and are formed by a piezoelectric transducer in the same manner as the first and the second ultrasound transducers 81 and 82 explained in the first embodiment described above.

The first ultrasound transducer 81B is attached to the bottom surface of the one vertical vibration enlargement unit 83B (the vertical vibration enlargement unit 83B attached under the probe 6 in FIG. 12). When an AC voltage of the first output Va1 (the frequency of the AC voltage is the same as the resonance frequency of the horizontal vibration of the probe 6) is applied to the first ultrasound transducer 81B under control of the controller 3, the first ultrasound transducer 81B expands and contracts in a direction along the central axis of the one vertical vibration enlargement unit 83B (a direction perpendicular to the central axis Ax).

The second ultrasound transducer 82B is attached to the bottom surface of the other vertical vibration enlargement unit 83B (the vertical vibration enlargement unit 83B attached to the left side of the probe 6 in FIG. 12 when observed from the distal end side of the treatment tool 2B). When an AC voltage of the first output Vb1 (the frequency of the AC voltage is the same as the resonance frequency of the horizontal vibration of the probe 6) is applied to the second ultrasound transducer 82B under control of the controller 3, the second ultrasound transducer 82B expands and contracts in a direction along the central axis of the other vertical vibration enlargement unit 83B (a direction perpendicular to the central axis Ax).

That is to say, in the modified example 1-2, the first and the second ultrasound transducers 81B and 82B are formed so as to generate vertical vibration (ultrasound vibration). The vertical vibration generated by the first and the second ultrasound transducers 81B and 82B is enlarged by each vertical vibration enlargement unit 83B and converted into horizontal vibration at a connection portion between the probe 6 and each vertical vibration enlargement unit 83B to generate horizontal vibration in the probe 6.

Therefore, even when the vibration unit 8B as described in the modified example 1-2 is employed, it is possible to perform the same joining control as the joining control (FIG. 9) explained in the first embodiment described above.

By employing the vibration unit 8B as described above, it is possible to increase the power of the horizontal vibration of the probe 6 as compared with a case in which the vibration unit 8 explained in the first embodiment described above is employed.

### Modified Example 1-3 of First Embodiment

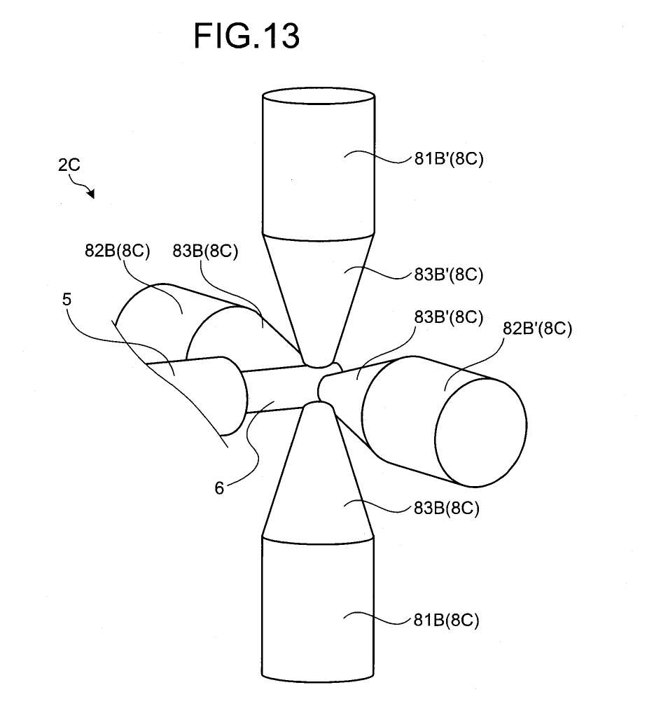

FIG. 13 is a diagram illustrating a modified example 1-3 of the first embodiment of the disclosure. Specifically, FIG. 13 is an enlarged schematic diagram of a part (one end side of the probe 6) of a treatment tool 2C according to the modified example 1-3.

In the modified example 1-2 described above, in the vibration unit 8B, only two vertical vibration enlargement units 83B (only the first and the second ultrasound transducers 81B and 82B) are attached to the probe 6. However, it is not limited to this.

For example, as in a vibration unit 8C (FIG. 13) according to the modified example 1-3, it is possible to employ a configuration in which two vertical vibration enlargement units 83B (the first and the second ultrasound transducers 81B and 82B) and two vertical vibration enlargement units 83B′ (a first and a second ultrasound transducers 81B′ and 82B′) are attached to the probe 6.

Here, a set of the first ultrasound transducer 81B′ and the vertical vibration enlargement unit 83B′ has the same configuration as that of a set of the first ultrasound transducer 81B and the vertical vibration enlargement unit 83B attached under the probe 6 in FIG. 13. The set of the first ultrasound transducer 81B′ and the vertical vibration enlargement unit 83B′ is attached to the probe 6 at a position rotationally symmetric by 180° with respect to the set of the first ultrasound transducer 81B and the vertical vibration enlargement unit 83B around the central axis Ax (at an upper position in FIG. 13).

The first ultrasound transducer 81B′ is applied with an AC voltage whose phase is opposite to that of an AC voltage applied to the first ultrasound transducer 81B by the first output Va1 under control of the controller 3.

Further, a set of the second ultrasound transducer 82B′ and the vertical vibration enlargement unit 83B′ has the same configuration as that of a set of the second ultrasound transducer 82B and the vertical vibration enlargement unit 83B attached to the left side of the probe 6 in FIG. 13 when observed from the distal end side of the treatment tool 2C. The set of the second ultrasound transducer 82B′ and the vertical vibration enlargement unit 83B′ is attached to the probe 6 at a position rotationally symmetric by 180° with respect to the set of the second ultrasound transducer 82B and the vertical vibration enlargement unit 83B around the central axis Ax (at a position in the right in FIG. 13 when observed from the distal end of the treatment tool 2C).

The second ultrasound transducer 82B′ is applied with an AC voltage whose phase is opposite to that of an AC voltage applied to the second ultrasound transducer 82B by the first output Vb1 under control of the controller 3.

Therefore, even when the vibration unit 8C as described in the modified example 1-3 is employed, it is possible to perform the same joining control as the joining control (FIG. 9) explained in the first embodiment described above except that the AC voltages applied to the first ultrasound transducers 81B and 81B′ (the second ultrasound transducers 82B and 82B′) have phases opposite to each other.

As described above, by increasing the numbers of ultrasound transducers and vertical vibration enlargement units, it is possible to increase power of the horizontal vibration in the probe 6.

### Second Embodiment

Next, a second embodiment of the disclosure will be described.

In the description below, the same reference numerals are given to the same components as those of the first embodiment described above and the detailed description thereof will be omitted or simplified.

In the medical treatment device 1 according to the first embodiment described above, AC voltages are respectively applied to the first and the second ultrasound transducers 81 and 82 by the first outputs Va1 and Vb1, so that the vibration direction D1 of the other end of the probe 6 is set to only the first direction with respect to a direction along the central axis Ax.

On the other hand, in the second embodiment, each output of AC voltages respectively applied to the first and the second ultrasound transducers 81 and 82 is sequentially changed to a first output, a second output, and a third output, so that the vibration direction of the other end of the probe 6 is sequentially changed to a first direction, a second direction, and a third direction. The second direction and the third direction are directions from the central axis Ax to the jaw portion 9 (the jaw portion main body 91) in the same manner as in the first embodiment.

The configuration of the medical treatment device according to the second embodiment is the same as that of the medical treatment device 1 explained in the first embodiment described above.

In the description below, only the joining control according to the second embodiment will be described.

Joining Control

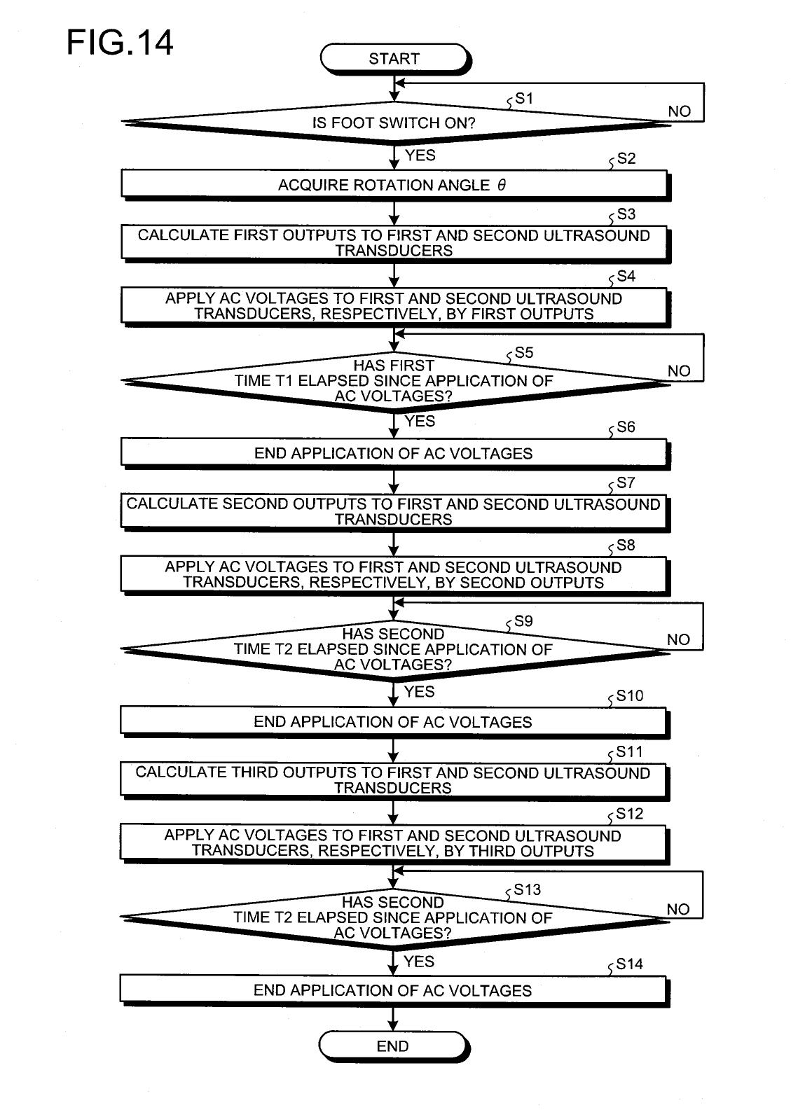

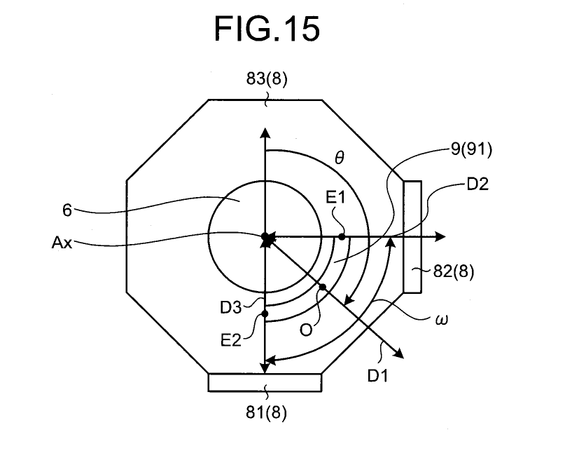

FIG. 14 is a flowchart illustrating the joining control according to the second embodiment of the disclosure. FIG. 15 is a diagram schematically illustrating horizontal vibration generated in the probe 6 by steps S8 and S12. Specifically, FIG. 15 is a diagram corresponding to FIG. 7B.

The joining control according to the second embodiment is different from the joining control explained in the first embodiment described above (FIG. 9) in that steps S7 to S14 are added as illustrated in FIG. 14.

Therefore, in the description below, only steps S7 to S14 will be described.

Step S7 is performed after step S6.

Specifically, in step S7, the output calculation unit 321 calculates a second output Va2 to the first ultrasound transducer 81 and a second output Vb2 to the second ultrasound transducer 82 from the following formulas (3) and (4) by using the rotation angle θ acquired in step S2.

$$\begin{matrix}Va 2 = Vo \times cos ( θ - \frac{ω}{2} ) & ( 3 ) \\ Vb 2 = Vo \times sin ( θ - \frac{ω}{2} ) & ( 4 )\end{matrix}$$

Here, in the above formulas (3) and (4), w means an angle representing expansion of the jaw portion main body 91 with respect to the central axis Ax as illustrated in FIG. 15. In other words, ω means an angle between a straight line connecting one end E1 in the width direction of the jaw portion main body 91 and the central axis Ax and a straight line connecting the other end E2 in the width direction of the jaw portion main body 91 and the central axis Ax.

After step S7, the transducer controller 322 drives the transducer application unit 31 and causes the transducer application unit 31 to apply AC voltages to the first and the second ultrasound transducers 81 and 82, respectively, by the second outputs Va2 and Vb2 (step S8).

When the AC voltages are applied to the first and the second ultrasound transducers 81 and 82, respectively, by the second outputs Va2 and Vb2, the respective first and the second ultrasound transducers 81 and 82 generates ultrasound vibration. Then, horizontal vibration is generated in the probe 6 by the ultrasound vibration generated by the respective first and the second ultrasound transducers 81 and 82. At this time, as illustrated in FIG. 15, a vibration direction D2 of the horizontal vibration (a vibration direction D2 at the other end of the probe 6) is set to a direction (a second direction) from the central axis Ax to the one end E1 in the width direction of the jaw portion main body 91 with respect to a direction along the central axis Ax regardless of the rotation angle θ of the jaw portion 9.

That is to say, each of the second outputs Va2 and Vb2 is an output that sets the vibration direction D2 of the other end of the probe 6 to the second direction with respect to a direction along the central axis Ax.

Subsequently, the transducer controller 322 monitors at all times whether or not a second time T2 has elapsed since the application of AC voltages in step S8 (step S9).

In the second embodiment, the second time T2 is set to a half of the first time T1. However, the second time T2 is not limited to a half of the first time T1, but may be any other time, for example, may be the same time as the first time T1.

When determining that the second time T2 has elapsed (step S9: Yes), the transducer controller 322 stops the drive of the transducer application unit 31 (ends the application of AC voltages to the first and the second ultrasound transducers 81 and 82) (step S10).

After step S10, the output calculation unit 321 calculates a third output Va3 to the first ultrasound transducer 81 and a third output Vb3 to the second ultrasound transducer 82 from the following formulas (5) and (6) by using the rotation angle θ acquired in step S2 (step S11).

$$\begin{matrix}Va 3 = Vo \times cos ( θ + \frac{ω}{2} ) & ( 5 ) \\ Vb 4 = Vo \times sin ( θ + \frac{ω}{2} ) & ( 6 )\end{matrix}$$

After step S11, the transducer controller 322 drives the transducer application unit 31 and the causes the transducer application unit 31 to apply AC voltages to the first and the second ultrasound transducers 81 and 82, respectively, by the third outputs Va3 and Vb3 (step S12).

When the AC voltages are applied to the first and the second ultrasound transducers 81 and 82, respectively, by the third outputs Va3 and Vb3, the respective first and the second ultrasound transducers 81 and 82 generates ultrasound vibration. Then, horizontal vibration is generated in the probe 6 by the ultrasound vibration generated by the respective first and the second ultrasound transducers 81 and 82. At this time, as illustrated in FIG. 15, a vibration direction D3 of the horizontal vibration (a vibration direction D3 at the other end of the probe 6) is set to a direction (a third direction) from the central axis Ax to the other end E2 in the width direction of the jaw portion main body 91 with respect to a direction along the central axis Ax regardless of the rotation angle θ of the jaw portion 9.

That is to say, each of the third outputs Va3 and Vb3 is an output that sets the vibration direction D3 of the other end of the probe 6 to the third direction with respect to a direction along the central axis Ax.

Subsequently, the transducer controller 322 monitors at all times whether or not the second time T2 has elapsed since the application of AC voltages in step S12 (step S13).

When determining that the second time T2 has elapsed (step S13: Yes), the transducer controller 322 stops the drive of the transducer application unit 31 (ends the application of AC voltages to the first and the second ultrasound transducers 81 and 82) (step S14).

The living tissues LT are joined by the processing described above.

According to the second embodiment described above, the effect described below is obtained in addition to the same effect as that of the first embodiment described above.

In the second embodiment, the outputs of the AC voltages applied to the first and the second ultrasound transducers 81 and 82 are sequentially changed to the first outputs Va1 and Vb1, the second outputs Va2 and Vb2, and the third outputs Va3 and Vb3. In other words, the vibration directions D1 to D3 are sequentially changed to the first direction (the direction from the central axis Ax to the center position O of the jaw portion 9 with respect to a direction along the central axis Ax), the second direction (the direction from the central axis Ax to the one end E1 in the width direction of the jaw portion main body 91 with respect to a direction along the central axis Ax), and the third direction (the direction from the central axis Ax to the other end E2 in the width direction of the jaw portion main body 91 with respect to a direction along the central axis Ax).

Therefore, it is possible to uniformly improve the joining strength of the entire living tissues LT sandwiched between the other end of the probe 6 and the jaw portion 9 (the jaw portion main body 91).

### Modified Example 2-1 of Second Embodiment

In the second embodiment described above, the vibration direction of the other end of the probe 6 is sequentially changed to the first direction, the second direction, and the third direction. However, it is not limited to this.

For example, it may be configured so that the vibration direction of the other end of the probe 6 is sequentially changed to two directions, which are the second direction and the third direction. In other words, in the joining control, steps S3 to S6 may be omitted.

In the living tissues LT sandwiched between the other end of the probe 6 and the jaw portion 9 (the jaw portion main body 91), when incising a portion pressed by the other end of the probe 6 and the jaw portion 9 (the jaw portion main body 91) along the first direction, it is not necessary to join the portion. In other words, portions may be joined which are respectively pressed along the second and the third directions by the other end of the probe 6 and the jaw portion 9 (the jaw portion main body 91). Therefore, in the case described above, by employing the configuration as described above, it is possible to avoid giving unnecessary vibration for joining.

Further, for example, it is possible to change the vibration direction of the other end of the probe 6 to a direction other than the first to the third directions if the direction is from the central axis Ax to the jaw portion 9 (the jaw portion main body 91) with respect to a direction along the central axis Ax.

### Modified Example 2-2 of Second Embodiment

The joining control (FIG. 14) explained in the second embodiment described above may be performed on the treatment tools 2A to 2C explained in the modified examples 1-1 to 1-3 described above.

### Third Embodiment

Next, a third embodiment of the disclosure will be described.

In the description below, the same reference numerals are given to the same components as those of the first embodiment described above and the detailed description thereof will be omitted or simplified.

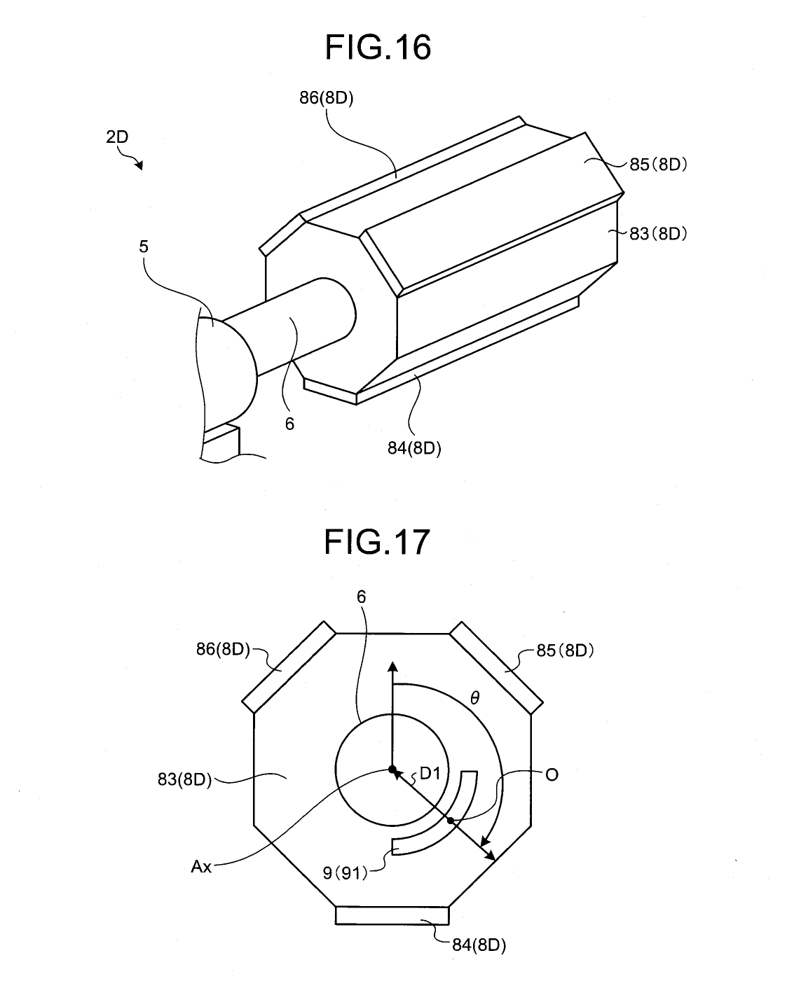

FIGS. 16 and 17 are diagrams schematically illustrating a treatment tool 2D according to the third embodiment of the disclosure. Specifically, FIG. 16 is an enlarged schematic diagram of a part (one end side of the probe 6) of a treatment tool 2D. Specifically, FIG. 17 is a schematic diagram of the probe 6, a vibration unit 8D, and the jaw portion 9 (the jaw portion main body 91) when observed from the distal end side of the treatment tool 2D along the central axis Ax.

In the medical treatment device 1 according to the first embodiment described above, the first and the second ultrasound transducers 81 and 82 are provided and the first and the second ultrasound transducers 81 and 82 are attached to positions 90° shifted from each other around the central axis Ax.

On the other hand, the medical treatment device according to the third embodiment employs a vibration unit 8D in which third to fifth ultrasound transducers 84 to 86 are attached to the horizontal vibration enlargement unit 83.

The third ultrasound transducer 84 has the same configuration as that of the first ultrasound transducer 81 explained in the first embodiment described above and is attached to the same position as the first ultrasound transducer 81 (is attached to a lower side surface in FIGS. 1 and 16 of the horizontal vibration enlargement unit 83).

Each of the fourth and the fifth ultrasound transducers 85 and 86 has the same configuration as that of the third ultrasound transducer 84. The fourth and the fifth ultrasound transducers 85 and 86 are respectively attached to two side surfaces 120° shifted around the central axis Ax with respect to the side surface where the third ultrasound transducer 84 is attached with respect to a direction along the central axis Ax among the eight side surfaces of the horizontal vibration enlargement unit 83. In other words, the side surfaces where the fourth and the fifth ultrasound transducers 85 and 86 are respectively attached are side surfaces 120° shifted from each other around the central axis Ax.

In the third embodiment, the output calculation unit 321 calculates a first output Vc1 to the third ultrasound transducer 84, a first output Vd1 to the fourth ultrasound transducer 85, and a first output Ve1 to the fifth ultrasound transducer 86 by the following formulas (7) to (9) by using the rotation angle θ of the jaw portion 9.

The reference position of the jaw portion 9 when the rotation angle sensor 20 detects the rotation angle θ is the same as the reference position explained in the first embodiment described above.

$$\begin{matrix}Vc 1 = \frac{Vo \times 2 \times ( sin ( θ1 ) + \frac{sin ( 3 \times θ1 )}{4} )}{\sqrt{3}} & ( 7 ) \\ Vd 1 = \frac{Vo \times 2 \times ( sin ( θ2 ) + \frac{sin ( 3 \times θ2 )}{4} )}{\sqrt{3}} & ( 8 ) \\ Ve 1 = \frac{Vo \times 2 \times ( sin ( θ3 ) + \frac{sin ( 3 \times θ3 )}{4} )}{\sqrt{4}} & ( 9 )\end{matrix}$$

Here, in the above formula (7), θ1 is θ+90°. In the above formula (8), θ2 is θ+210°. In the above formula (9), θ3 is θ+330°.

When AC voltages are applied to the third to the fifth ultrasound transducers 84 to 86, respectively, by the first outputs Vc1, Vd1, and Ve1, the horizontal vibration is generated in the same manner as in the first embodiment described above by ultrasound vibration generated by the third to the fifth ultrasound transducers 84 to 86. At this time, as illustrated in FIG. 17, a vibration direction D1 of the horizontal vibration (a vibration direction D1 at the other end of the probe 6) is set to a direction from the central axis Ax to the center position O of the jaw portion 9 (a first direction) with respect to a direction along the central axis Ax regardless of the rotation angle θ of the jaw portion 9.

That is to say, each of the first outputs Vc1, Vd1, and Ve1 is an output that sets the vibration direction D1 of the other end of the probe 6 to the first direction with respect to a direction along the central axis Ax.

Therefore, even when the vibration unit 8D as described in the third embodiment is employed, it is possible to perform the same joining control as the joining control (FIG. 9) explained in the first embodiment described above except for the first outputs Vc1, Vd1, and Ve1 to the third to the fifth ultrasound transducers 84 to 86.

According to the third embodiment described above, the effect described below is obtained in addition to the same effect as that of the first embodiment described above.

In the third embodiment, the third to the fifth ultrasound transducers 84 to 86 respectively attached to positions 120° shifted from each other around the central axis Ax are provided, and the AC voltages of the first outputs Vc1, Vd1, and Ve1 calculated by the formulas (7) to (9) are respectively applied to the third to the fifth ultrasound transducers 84 to 86.

Therefore, according to the third embodiment, it is possible to increase the power of the horizontal vibration of the probe 6 as compared with the configuration explained in the first embodiment described above.

### Fourth Embodiment

Next, a fourth embodiment of the disclosure will be described.

In the description below, the same reference numerals are given to the same components as those of the first embodiment described above and the detailed description thereof will be omitted or simplified.

The medical treatment device 1 according to the first embodiment described above applies only the ultrasound vibration (ultrasound energy) to the living tissues LT sandwiched between the other end of the probe 6 and the jaw portion 9 (the jaw portion main body 91).

On the other hand, a medical treatment device according to the fourth embodiment is configured to apply high frequency energy in addition to the ultrasound vibration to the living tissues LT.

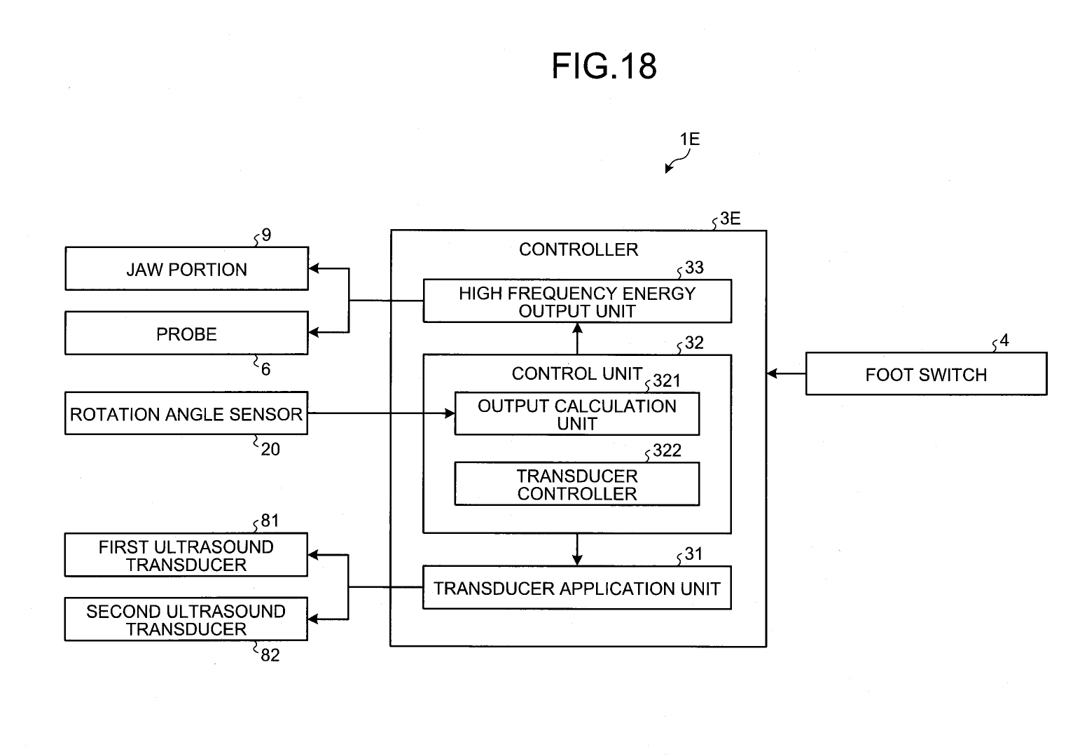

FIG. 18 is a block diagram illustrating a configuration of a controller 3E in a medical treatment device 1E according to the fourth embodiment of the disclosure.

The jaw portion 9 and the probe 6 according to the fourth embodiment have a function as an electrode that applies high frequency energy to the sandwiched living tissues LT.

In the controller 3E according to the fourth embodiment, as illustrated in FIG. 18, a high frequency energy output unit 33 is added to the controller 3 (FIG. 8) explained in the first embodiment described above.

The high frequency energy output unit 33 is electrically connected to each of the jaw portion 9 and the probe 6 and supplies high frequency power to the jaw portion 9 and the probe 6 under control of the control unit 32.

The timing of applying the high frequency energy to the living tissues LT may be before applying the ultrasound vibration (before steps S2 to S4), after applying the ultrasound vibration (after step S6), or at the same time as applying the ultrasound vibration.

According to the fourth embodiment described above, the effect described below is obtained in addition to the same effect as that of the first embodiment described above.

The medical treatment device 1E according to the fourth embodiment applies the ultrasound vibration and the high frequency energy to the living tissues LT.

Therefore, it is possible to improve the joining strength of the living tissues LT by combining different types of energies as in the fourth embodiment.

### Modified Example 4-1 of Fourth Embodiment

It is possible to employ the configuration explained in the fourth embodiment described above on the configurations explained in the second and the third embodiments and the modified examples 1-1 to 1-3, 2-1, and 2-2 described above.

### Fifth Embodiment

Next, a fifth embodiment of the disclosure will be described.

In the description below, the same reference numerals are given to the same components as those of the first embodiment described above and the detailed description thereof will be omitted or simplified.

The medical treatment device 1 according to the first embodiment described above applies only the ultrasound vibration (ultrasound energy) to the living tissues LT sandwiched between the other end of the probe 6 and the jaw portion 9 (the jaw portion main body 91).

On the other hand, a medical treatment device according to the fifth embodiment is configured to apply thermal energy in addition to the ultrasound vibration to the living tissues LT.

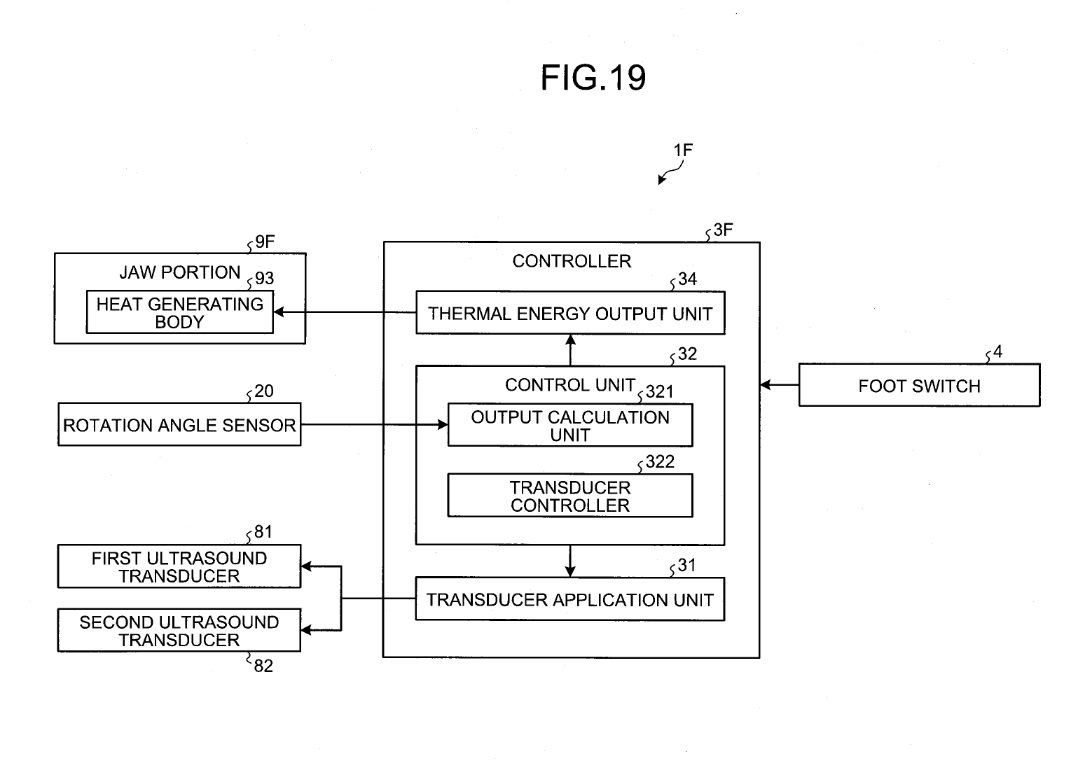

FIG. 19 is a block diagram illustrating a configuration of a controller 3F in a medical treatment device 1F according to the fifth embodiment of the disclosure.

In a jaw portion 9F according to the fifth embodiment, as illustrated in FIG. 19, a heat generating body 93 is added to the jaw portion 9 explained in the first embodiment described above.

The heat generating body 93 is a member which is attached to the jaw portion main body 91 and generates heat to heat up the jaw portion main body 91 under control of the controller 3F. In other words, the heat generating body 93 is a member which applies thermal energy to the living tissues LT through the jaw portion main body 91.

Although not illustrated specifically in FIG. 19, the heat generating body 93 includes a heat generating sheet where a heat generating pattern is formed by vapor deposition or the like on a sheet-like substrate formed from an insulating material and which generates heat when a voltage is applied to (a current is flown through) the heat generating pattern. However, the heat generating body 93 is not limited to the heat generating sheet, but the heat generating body 93 may include a plurality of heat generating chips and generate heat when a current is drawn through the plurality of heat generating chips (for example, see JP 2013-106909 A for the above technique).

In the controller 3F according to the fifth embodiment, as illustrated in FIG. 19, a thermal energy output unit 34 is added to the controller 3 (FIG. 8) explained in the first embodiment described above.

The thermal energy output unit 34 is electrically connected to the heat generating body 93 and applies a voltage to (flows a current through) the heat generating body 93 under control of the control unit 32.

The timing of applying the thermal energy to the living tissues LT may be before applying the ultrasound vibration (before steps S2 to S4), after applying the ultrasound vibration (after step S6), or at the same time as applying the ultrasound vibration.

According to the fifth embodiment described above, the effect described below is obtained in addition to the same effect as that of the first embodiment described above.

The medical treatment device 1F according to the fifth embodiment applies the ultrasound vibration and the thermal energy to the living tissues LT.

Therefore, it is possible to improve the joining strength of the living tissues LT by combining different types of energies as in the fifth embodiment.

### Modified Example 5-1 of Fifth Embodiment

It is possible to employ the configuration explained in the fifth embodiment described above on the configurations explained in the second to the fourth embodiments and the modified examples 1-1 to 1-3, 2-1, 2-2, and 4-1 described above.

Further, regarding the heat generating body 93, a configuration may be employed in which the heat generating body 93 is attached to the jaw portion main body 91 and the other end of the probe 6, and a configuration may be employed in which the heat generating body 93 is attached to only the other end of the probe 6.

### Other Embodiments

While the embodiments for implementing the disclosure have been described, the disclosure should not be limited by only the first to the fifth embodiments and the modified examples 1-1 to 1-3, 2-1, 2-2, 4-1, and 5-1 described above.

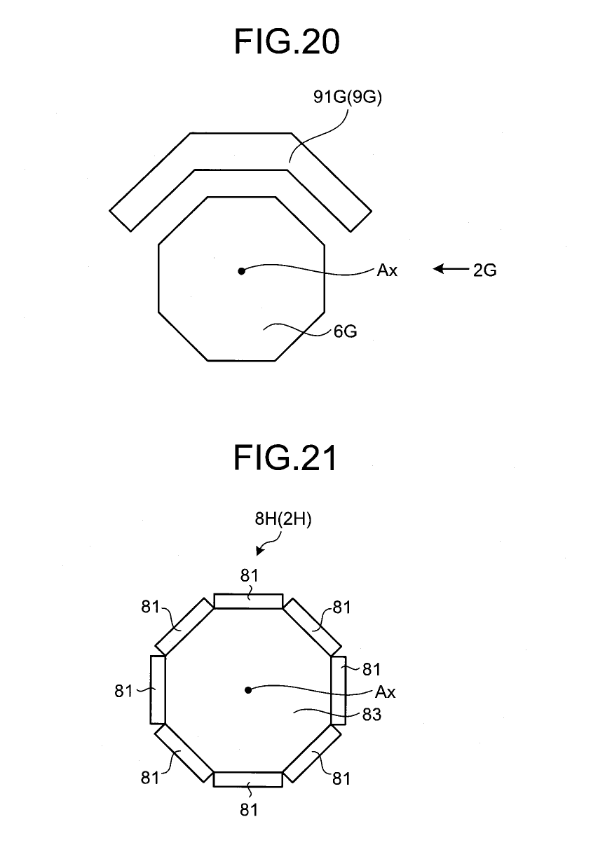

FIGS. 20 and 21 are diagrams illustrating a modified example of the first to the fifth embodiments of the disclosure.

In the first to the fifth embodiments and the modified examples 1-1 to 1-3, 2-1, 2-2, 4-1, and 5-1 described above, the probe 6 has a circular shape in cross-sectional view. Further, the jaw portion main body 91 has an arc shape in cross-sectional view following the outer circumferential surface of the probe 6.

The cross-sectional shapes of the probe 6 and the jaw portion main body 91 are not limited to the cross-sectional shapes described above, but may be cross-sectional shapes as those of a probe 6G and a jaw portion main body 91G (a jaw portion 9G) in a treatment tool 2G illustrated in FIG. 20.

Specifically, the cross-sectional shape of the probe 6G is a regular octagonal shape as illustrated in FIG. 20. The cross-sectional shape of the jaw portion main body 91G is a shape that extends in parallel along three side surfaces adjacent to each other of the eight side surfaces of the probe 6G following the outer circumferential surface of the probe 6G.

In the first to the fifth embodiments, the modified examples 1-1 to 1-3, 2-1, 2-2, 4-1, and 5-1, and FIG. 20 described above, the cross-sectional shapes of the jaw portion main bodies 91 and 91G are shapes following the cross-sectional shapes of the probes 6 and 6G. However, they are not limited to these, and the cross-sectional shapes of the jaw portion main bodies 91 and 91G and the cross-sectional shapes of the probes 6 and 6G need not correspond to each other. For example, a jaw portion main body having a flat plate shape instead of an arc shape in cross-sectional view following the outer circumferential surface of the probe 6 may be combined with the probe 6 having a circular shape in cross-sectional view.

In the first to the fifth embodiments and the modified examples 1-1 to 1-3, 2-1, 2-2, 4-1, and 5-1 described above, the ultrasound transducer according to the disclosure is formed by a piezoelectric transducer. However, it is not limited to this, and the ultrasound transducer may be formed by using a magnetostrictive transducer.

In the first and the third embodiments and the modified example 1-1 described above, the ultrasound transducer is attached to two to four side surfaces of the eight side surfaces of the horizontal vibration enlargement unit 83. However, it is not limited to this. For example, as in a treatment tool 2H (vibration unit 8H) illustrated in FIG. 21, the ultrasound transducer (the first ultrasound transducer 81 in the example of FIG. 21) may be attached to five or more side surfaces or all the side surfaces.

In the first to the fifth embodiments and the modified examples 1-1 to 1-3, 2-1, 2-2, 4-1, and 5-1 described above, the jaw portion 9 is opened and closed with respect to the probe 6. However, it is not limited to this, and it is possible to employ a configuration in which the probe 6 and the jaw portion 9 are opened and closed by moving both the probe 6 and the jaw portion 9 and a configuration in which the probe 6 is opened and closed with respect to the jaw portion 9.

The flow of the joining control is not limited to the order of the processing in the flowcharts (FIGS. 9 and 14) explained in the first to the fifth embodiments and the modified examples 1-1 to 1-3, 2-1, 2-2, 4-1, and 5-1 described above, and the order of the processing may be changed within a range without contradiction.

### REFERENCE SIGNS LIST

The medical treatment device of the disclosure produces effects that the operability can be improved and also the joining strength of the living tissues can be improved.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical treatment device comprising:
a vibration unit including a plurality of ultrasound transducers, each ultrasound transducer being configured to generate ultrasound vibration;
a probe which extends linearly and where the vibration unit is attached to one end of the probe and the ultrasound vibration generated by each of the ultrasound transducers is transmitted from the one end to other end of the probe;
a jaw portion configured to: sandwich living tissues between the jaw portion and the other end of the probe by moving relative to the probe; and rotate around a central axis of the probe;
a rotation angle sensor configured to detect a rotation angle of the jaw portion around the central axis; and
a controller configured to: calculate outputs, which respectively drive the ultrasound transducers, based on the rotation angle of the jaw portion; and drive each of the ultrasound transducers by each of the calculated outputs,
wherein each output is an output that sets a direction of vibration of the other end caused by ultrasound vibration generated by each of the ultrasound transducers to a direction from the central axis to the jaw portion with respect to a direction along the central axis.

2. The medical treatment device according to claim 1, wherein
each output includes a first output that sets the vibration direction of the other end to a first direction from the central axis to a center position of the jaw portion with respect to the direction along the central axis.

3. The medical treatment device according to claim 1, wherein
each output includes: a second output that sets the vibration direction of the other end to a second direction from the central axis to one position in the jaw portion with respect to the direction along the central axis; and a third output that sets the vibration direction of the other end to a third direction from the central axis to other position different from the one position in the jaw portion with respect to the direction along the central axis, and
the controller is configured to sequentially drive each of the ultrasound transducers by the second output and the third output.

4. The medical treatment device according to claim 3, wherein
the one position and the other position in the jaw portion are respectively located on both sides of the center position of the jaw portion with respect to the direction along the central axis.

5. The medical treatment device according to claim 4, wherein
each output further includes a first output that sets the vibration direction of the other end to a first direction from the central axis to the center position of the jaw portion with respect to the direction along the central axis, and
the controller is configured to sequentially drive each of the ultrasound transducers by the first output, the second output, and the third output.

6. The medical treatment device according to claim 1, wherein
the controller is further configured to apply high frequency energy to the living tissues sandwiched between the probe and the jaw portion.

7. The medical treatment device according to claim 1, wherein
at least one of the jaw portion and the probe includes a heat generating body configured to generate heat when a current is flown through the heat generating body, and
the controller is further configured to apply thermal energy to the living tissues sandwiched between the probe and the jaw portion when a current is flown through the heat generating body.
